(19) United States
(12) Patent Application Publication
(10) Pub. No.: US 2011/0269839 A1
(43) Pub. Date: Nov. 3, 2011

(54) ANTI-SNORING COMPOSITION CONTAINING A TERMOGELLING POLYMER

(75) Inventors: Marc Dolatkhani, Cestas (FR); Anne Pagnoux, Le Barp (FR)

(73) Assignee: POLYMEREXPERT SA, Pessac (FR)

(21) Appl. No.: 13/143,122

(22) PCT Filed: Jan. 7, 2010
(86) PCT No.: PCT/FR2010/050021

§ 371 Date: Jul. 1, 2011

(30) Foreign Application Priority Data
Jan. 7, 2009 (FR) 0950057

(51) Int. Cl. A61K 31/13; A61P 9/00; A61P 29/00; A61P 11/00; A61P 11/02

(52) U.S. Cl. 514/638

(57) ABSTRACT

The invention relates to a composition for treating and/or preventing snoring, comprising at least one thermogelling polymer that includes water-soluble units as well as units having a lower critical demixing temperature in water. Said composition has the specific feature of being liquid in the packaging thereof and becoming a gel upon contacting mucus membranes at 37° C. Thermogelling makes it possible to precisely target the area to be treated while avoiding any running or migration of the material, and makes the sprayed composition difficult to wash off, thus increasing the duration of the presence thereof in the treatment area.

The invention relates to the development of a thermogelling formulation for preventing and/or treating snoring, which can be administered by spraying via the nasal or buccal routes.

Snoring is associated with respiratory problems during sleep. The sound produced, which can reach 100 decibels, results from vibration of the nasopharyngeal tissues. The air entering through the nose and mouth must flow through a narrow passage between the tongue, the soft palate, the uvula and the wall of the pharynx. During sleep, the muscles are relaxed and tend to sag, causing further narrowing of the passage. These soft structures press against one another and prevent the air passing through freely. The vibration of the tissues is what causes snoring. Snoring also occurs when there is narrowing of the nasal passages.

Snoring is particularly common in adults over 40 and is promoted by fatigue, being overweight, or taking alcohol or certain medicaments. It is estimated that 60% of men and 40% of women over 40 snore occasionally and that about 25% of men and 15% of women suffer from severe pathological snoring called rhonchopathy.

The treatments recommended can be of various kinds:
sleep on one's side or on one's stomach: this increases the space for the passage of air,
rest and avoid drinking alcohol or taking certain medicaments,
lose weight: reduction in volume of the base of the tongue and of the pharyngeal tissues improves the passage of air,
use systems that promote the passage of air (nostril expander, oropharyngeal cannula, mandibular advancement orthosis, etc.),
surgery: pharyngoplasty consisting of sectioning the edge of the soft palate and the uvula;
use of buccal and nasal sprays, whose action consists of lubricating the soft tissues during sleep, thus preventing the vibrations that are the cause of snoring.

The products proposed in sprays developed to date and available on the market have limited efficacy on snoring on account of their composition.

In fact, this type of product must provide high lubricating power and considerable adhesiveness on the tissues to ensure its effectiveness throughout sleep.

Two types of composition have mainly been described in the literature, namely:

a) oil-based preparations

The use of edible oils makes it possible to lubricate the mucosae. However, the duration of action of this type of product is very short owing to the low affinity of the oil (hydrophobic) for the hydrophilic mucosae. In fact, the oil droplets are very quickly carried away to the stomach, aided by swallowing. Another drawback in using this type of preparation is the risk of oil-aspiration pneumopathy connected with passage of oil droplets into the lungs.

b) preparations based on polysaccharides or acrylic derivatives. The use of polysaccharide in the composition of anti-snoring products as muco-adhesive agent and/or lubricant is described, for example, in patent applications FR 2 859 105, WO 2006/042926 and WO 2007/138224. These polysaccharides include hyaluronic acid, which has been used for years as a lubricant in the ophthalmic field and whose muco-adhesive properties are described in detail in the literature. Other polysaccharides, such as carrageenans, are used solely for their muco-adhesive properties and are combined with lubricants.

Like all polysaccharides, the acrylic derivatives cited are water-soluble, and are quickly washed away by the flow of saliva, which constitutes the main drawback of this approach. This point is all the more true as these polymers are used in small proportions because they cause considerable increase in viscosity of the formulations (even when present at low concentration), making spraying of the products more difficult. The major drawback of these preparations concerns the ease with which they are quickly washed away by the flow of saliva.

The invention therefore relates to a sprayable composition intended for effective and long-lasting prevention and/or treatment of snoring, comprising at least one thermogelling polymer displaying both muco-adhesive and lubricating properties. The approach consists of spraying a product that is liquid at room temperature by the nasal or buccal route, which gels on contact with the mucosae, ensuring that the product is maintained throughout sleep.

The invention also relates to the use of at least one thermogelling polymer in a composition for preventing and/or treating snoring.

More particularly, the invention relates to a composition comprising at least one thermogelling polymer in aqueous solution, said thermogelling polymer comprising, on the one hand, water-soluble units and, on the other hand, units having a lower critical solution temperature in water, which are present at a concentration by weight of less than or equal to 10% in said aqueous solution, for preventing and/or treating snoring.

By “thermogelling polymers” is meant polymers which, at low concentration in water, are in liquid form (low viscosity) at room temperature and which gel at body temperature: the phenomenon of thermal gelling being completely reversible.

Advantageously, the aqueous solution of thermogelling polymer can be used for the purposes of the invention has a viscosity below 0.1 Pa·s under 10 s⁻¹ of shearing at room temperature, and a viscosity above 0.1 Pa·s, preferably above 0.3 Pa·s under 10 s⁻¹ of shearing in contact with the mucosae, at body temperature.

By “ambient temperature” is meant a temperature of the order of 18 to 25° C.

The concentration by weight of thermogelling polymer in aqueous solution is less than or equal to 10%, preferably between 2 and 10%. It should be noted that the viscosity values of the aqueous solution of thermogelling polymer are given above for shearing of 10 s⁻¹ (shearing applied during spraying). Said polymer being shear-thinning, its viscosity decreases with shearing. However, as shearing is practically zero in the nasal or buccal passages, the effective viscosity on contact with the nasal or buccal mucosae is much higher, permitting said aqueous solution of thermogelling polymer to be in the form of gel.

The thermogelling polymers which can be used for the purposes of the invention comprise, on the one hand, water-soluble units and, on the other hand, units having a lower critical solution temperature in water (defined by the abbreviation “LCST”). Below the LCST, the polymer is completely soluble in water, whereas above this temperature the LCST portions aggregate and lose their solubility in water, thus forming crosslinks between the polymer chains. The polymer then becomes like a three-dimensional network, leading to formation of a gel. As this association of LCST chains within the hydrophobic micro-domains above the demixing temperature is of a physical nature, the phenomenon of gelling is completely reversible.

This property of gelling with temperature is only possible if the concentration of polymer is greater than the critical micelle concentration to permit interactions between the LCST units carried by different polymer chains.

The polymers used in the invention can either be block polymers or graft polymers, comprising water-soluble units on the one hand, and LCST units on the other hand.

These water-soluble units can be polymers of natural origin or of synthetic origin obtained by chain polymerization or by polycondensation.

The LCST units will preferably be selected from:
polyethers such as polypropylene oxide) PPO and its random and block copolymers with poly(ethylene oxide)
N-substituted derivatives of acrylamide such as poly(N-isopropylacrylamide) or poly(N-ethylacrylamide)
polyvinylcaprolactam and copolymers of vinylcaprolactam.

The thermogelling polymers that may be suitable for the invention can be selected for example from those described in the following patent applications and patents: FR2694939, FR2788008, FR2820976, FR2856923, GB2408510, EP0629649, EP 1307501, EP1407791, WO97/00275, WO98/06438, WO98/29487, WO98/48768, WO98/50005, WO00/00222, WO00/07603, WO00/35961, WO00/38851, WO01/41735, WO02/032560, WO02/076392, WO03/008462, WO03/106536, WO04/006872, US2003/0099709, U.S. Pat. No. 6,645,476, U.S. Pat. No. 6,689,855, U.S. Pat. No. 6,689,856, U.S. Pat. No. 6,870,012 and U.S. Pat. No. 6,878,754.

The thermogelling polymers that are particularly suitable for the compositions for combating snoring according to the invention are the polyurethanes having polyethylene oxide-b-propylene oxide-b-ethylene oxide) (PEO-b-PPO-b-PEO) groups such as those described in patents and patent applications FR2840907, EP692506, EP1407791, WO 03 106536, U.S. Pat. No. 7,339,013.

These polyurethanes are obtained by polycondensation of diisocyanates and of PEO-b-PPO-b-PEO triblock diols that are heat-sensitive in a non-anhydrous medium and can comprise urea groups and/or allophanates. These polymers, known by the name ExpertGel® and marketed by the company PolymerExpert offer numerous advantages relative to the polymers of the Poloxamer® type and to conventional aqueous-phase viscosity improvers, in particular:
a low concentration of polymers is required for a large increase in viscosity as a function of temperature,
they have good lubricating properties,
they are muco-adhesive,
they display the same thermogelling behaviour for a pH range from 1 to 12,
they are compatible with salts, surfactants and most pharmaceutical and cosmetic excipients,
they display shear-thinning behaviour, making spraying of them possible at elevated temperature (in an overheated bathroom, in full sun etc.),
they have a gelling temperature that can be adjusted accurately between 10 and 60° C., they are non-toxic products, which can be used without restriction in applications in contact with the skin and mucosae.

The solutions of polymers of the ExpertGel® type are therefore particularly suitable for the intended application.

The first advantage relates to the method of delivery by spraying. In fact, the composition containing the thermogelling polymers is liquid in its packaging at room temperature and becomes a gel on contact with the mucosae at 37° C. Thermal gelation permits precise targeting of the zone to be treated, avoiding any running or migration of the product and makes the sprayed product resistant to being washed away, increasing the duration of its presence in the treatment zone.

The second advantage in using these polymers is connected with their exceptional muco-adhesive properties, as shown by the works of Pr. Jean-Louis Grossiord et al. in J. Drug Del. Sci. Tech., 16(1) 59-4 2006. The authors compared the adhesion of two types of ExpertGel® polymer, namely EG 30 and EG 40, with that of CARBOPOL 974P, both on synthetic substrates and on the intestinal mucosae of the rat. CARBOPOL 974, which is a partially neutralized polyacrylic acid, is known in the pharmaceutical industry as a reference polymer owing to its bioadhesive properties. The results show that the peel strength at 37° C. under their operating conditions is 0.2 N for CARBOPOL 974P whereas it is 0.7 N for EG 35 and 0.3 for EG 40. They therefore conclude that the two types of EG polymers, and more particularly EG35, have better muco-adhesive properties than CARBOPOL 974P at 37° C., which makes these thermogelling polymers interesting products for the development of cosmetic and pharmaceutical formulations in contact with the skin and mucosae.

The lubricating capacity of these polymers obviously constitutes another advantage in using them for the anti-snoring application. The thermogelling polymers, in particular the thermogelling polymers constituted essentially by PEO-b-PPO-b-PEO block copolymers, display surface-active properties. The lubricating properties of “soaps” have been known for a very long time. Many studies have demonstrated in particular the sliding properties resulting from hydration of the PEO units; the phenomenon described can be likened to a process of “aquaplaning”. Inspired by these works, the applicant has also developed coatings based on ExpertGel® in the biomedical field to facilitate the placement of implants or probes. Devices coated with polymers of the ExpertGel® type display properties of considerable sliding in contact with the mucosae and do not cause any problems at the time of placement. One example concerns urinary-tract probes which, when coated with a layer of ExpertGel®, slide perfectly and can be put in place without any discomfort for the patient.

To summarize, the thermogelling polymers which can be used for the purposes of the invention permit:

1) easy application by spraying,

2) treatment of a precise zone because of immediate gelling of the sprayed solution,

3) good lubrication of the zone treated,

4) high efficacy of treatment owing to their muco-adhesive properties and high resistance of the gel to washing-away (little solution of the gel by the flow of saliva).

Advantageously, the anti-snoring compositions according to the invention are therefore suitable for spraying, by the nasal or buccal route, they display muco-adhesive properties enabling them to resist being washed away by the flow of saliva and they display lubrication properties of the mucosae. These muco-adhesive and lubricating properties are better than those of the commercially available products.

Another advantage connected with the use of thermogelling polymers is the possibility of controlled release of an active ingredient from the composition containing them. The polymer solution containing the active ingredient gels at 37° C. The active ingredient is coated by the gel that has formed and can diffuse in a controlled manner.

The invention therefore also relates to a composition for preventing and/or treating snoring comprising a thermogelling polymer, as described above, containing an active ingredient for controlled release.

For example, the release of a decongestant, of an anti-inflammatory or of an agent for treating or preventing snoring, for example a vasoconstrictor may be mentioned, such as extract of Ruscus aculeates, which could be encapsulated by the thermogelling polymer and released with controlled pharmacokinetics during the period of sleep.

By “decongestant” is meant a product that promotes the elimination of excess blood, and by “vasoconstrictor” is meant a product that reduces the calibre of a vessel by contraction of its muscle fibres.

As mentioned in patents and patent applications FR 2840907, EP1407791, EP692506, WO03106536 and U.S. Pat. No. 7,339,013, the manufacture of thermogelling polyurethanes can be adapted to the required application. Thus, the gelling temperature, the polarity of the macromolecule, and the variation of viscosity can be adjusted as a function of the reactants and the synthesis conditions, permitting efficient coating of the active ingredient and controlled release thereof.

Some of the parameters can also be adjusted by formulating these products with other additives.

Thus, the anti-snoring compositions according to the invention can optionally comprise one or more additives selected from:
electrolytes, for example sodium chloride, magnesium chloride, sodium fluoride etc. The presence of salts in the mixture promotes demixing and lowers the gelling temperature; said electrolytes can be present in the composition, for example, up to 15 wt. %;
lubricants, such as glycerol for example. Glycerol also has the effect of lowering the gelling temperature and its addition can increase the lubrication properties of the anti-snoring composition. Said lubricants can be present in the composition, for example, up to 30 wt. %;
alcohols, for example alcohols such as ethanol or isopropanol, as preservatives of the solution.

Said alcohol will preferably be used at a concentration below 10% in the formulation mixture, in particular of the order of 5%.
non-ionic surfactants, for example Tween® or Brij®; said lubricants can be present in the composition, for example, up to 10 wt. %.

The addition of a non-ionic surfactant has a dual effect. Firstly a lowering of gelling temperature is observed. More surprisingly, we observe a synergistic effect between the surfactant and the thermogelling polymer, leading to an even greater increase in viscosity as a function of temperature.
polysaccharides, for example hyaluronic acid, alginates, in particular sodium alginate, xanthans etc.

The addition of a very small amount of polysaccharide, in particular less than 5 wt. %, makes it possible to adjust the viscosity of the anti-snoring composition at room temperature. The polysaccharide is then used as conventional viscosity improver of the formulation.
active agents, for example agents having an anti-inflammatory or vasoconstrictive action.

The invention is illustrated by the following non-limitative examples. Unless stated otherwise, percentages are expressed by weight.

### EXAMPLE 1

The following anti-snoring composition was prepared:

| EG56 SEC (ExpertGel ®) | 3 | g | 6% | |
|---|---|---|---|---|
| Ethanol | 2.5 | g | 5% | |
| Menthol | 0.05 | g | 0.1% | |
| Mineral water | 44.4 | g | q.s. | 100% |

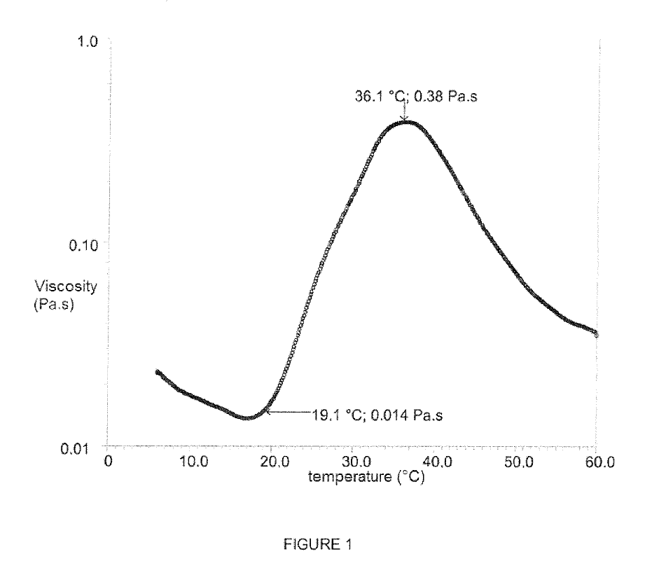

The thermogelling polymer EG56SEC (CAS number 93665-35-1) is a polyurethane-urea based on PEO-PPO-PEO containing about 75% of PEO, the INCI (International Nomenclature of Cosmetic Ingredients) designation of which is BIS-Methoxy PEG-13 PEG-438/PPG-110 SMDI Copolymer. The flow viscosity curve under shear at 10 s⁻¹ of the composition of Example 1 as a function of temperature is shown in FIG. 1.

### EXAMPLE 2

The following anti-snoring composition was prepared:

| EG56 SEC (ExpertGel ®) | 3 | g | 6% | |
|---|---|---|---|---|
| Ethanol | 2.5 | g | 5% | |
| Menthol | 0.05 | g | 0.1% | |
| Hyaluronic acid | 0.1 | g | 0.2% | |
| Mineral water | 44.35 | g | q.s. | 100% |

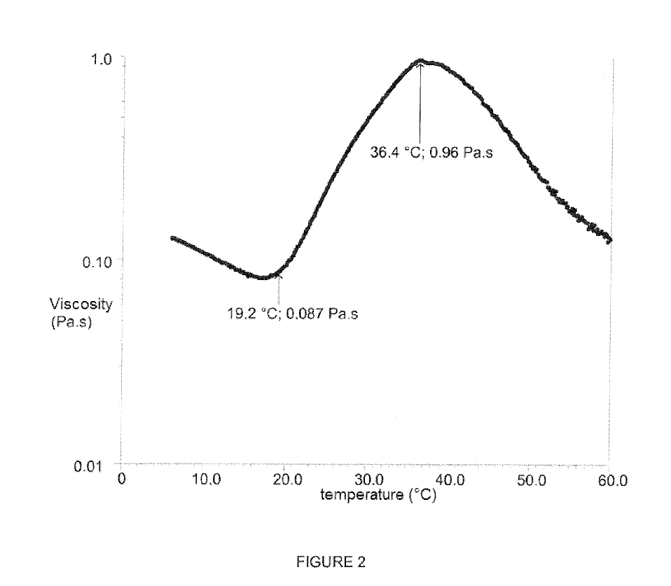

The flow viscosity curve under shear at 10 s⁻¹ of the composition of Example 2 as a function of temperature is shown in FIG. 2.

### EXAMPLE 3

The following anti-snoring composition was prepared:

| EG56 SEC (ExpertGel ®) | 3 | g | 6% | |
|---|---|---|---|---|
| Ethanol | 2.5 | g | 5% | |
| Menthol | 0.05 | g | 0.1% | |
| Hyaluronic acid | 0.1 | g | 0.2% | |
| Tween 20 ® | 0.05 | g | 0.1% | |
| Mineral water | 44.4 | g | q.s. | 100% |

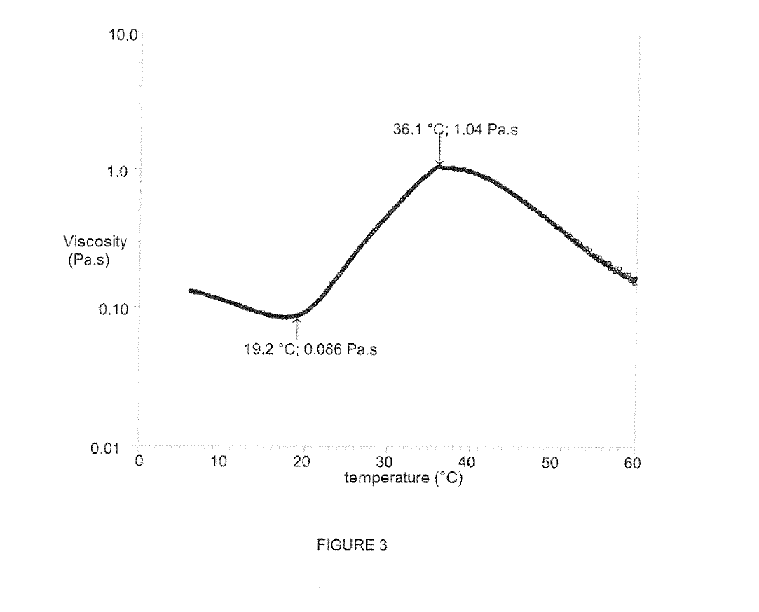

The flow viscosity curve under shear at 10 s⁻¹ of the composition of Example 3 as a function of temperature is shown in FIG. 3.

### EXAMPLE 4

The following anti-snoring composition was prepared:

| EG56 SEC (ExpertGel ®) | 3 | g | 6% | |
|---|---|---|---|---|
| Ethanol | 2.5 | g | 5% | |
| Menthol | 0.05 | g | 0.1% | |
| Hyaluronic acid | 0.1 | g | 0.2% | |
| Tween 20 ® | 0.05 | g | 0.1% | |
| Chlorhexidine | 0.05 | g | 0.1% | |
| Mineral water | 44.20 | g | q.s. | 100% |

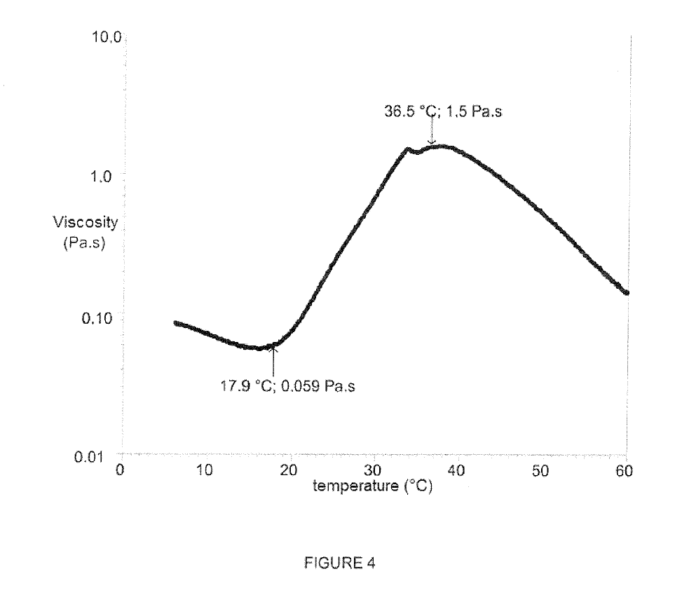

The flow viscosity curve under shear at 10 s⁻¹ of the composition of Example 4 as a function of temperature is shown in FIG. 4.

### EXAMPLE 5

The following anti-snoring composition was prepared:

| EG 230 (ExpertGel ®) | 3.5 | g | 7% | |
|---|---|---|---|---|
| Ethanol | 2.5 | g | 5% | |
| Sodium chloride | 0.45 | g | 0.9% | |
| Menthol | 0.05 | g | 0.1% | |
| Tween 20 ® | 0.15 | g | 0.3% | |
| Mineral water | 43.35 | g | q.s. | 100% |

The thermogelling polymer EG230 (CAS number 93665-35-1) is a branched polyurethane containing PEO-b-PPO-b-PEO units, the INCI (International Nomenclature of Cosmetic Ingredients) designation of which is BIS-Methoxy PEG-13 PEG-502/PPG-57 SMDI Copolymer.

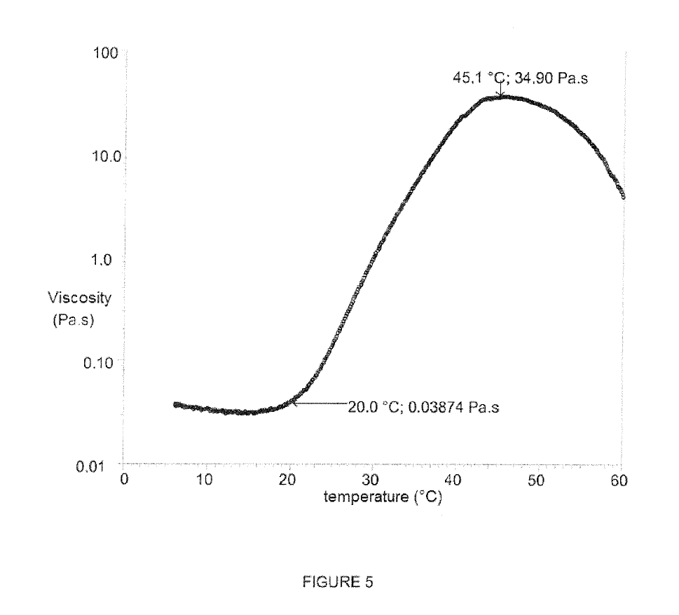

The flow viscosity curve under shear at 10 s⁻¹ of the composition of Example 5 as a function of temperature is shown in FIG. 5.

### EXAMPLE 6

The following anti-snoring composition was prepared:

| EG 230 (ExpertGel ®) | 3.5 | g | 7% | |
|---|---|---|---|---|
| Ethanol | 2.5 | g | 5% | |
| Hyaluronic acid | 0.1 | g | 0.2% | |
| Sodium chloride | 0.45 | g | 0.9% | |
| Menthol | 0.05 | g | 0.1% | |
| Tween 20 ® | 0.15 | g | 0.3% | |
| Mineral water | 43.25 | g | q.s. | 100% |

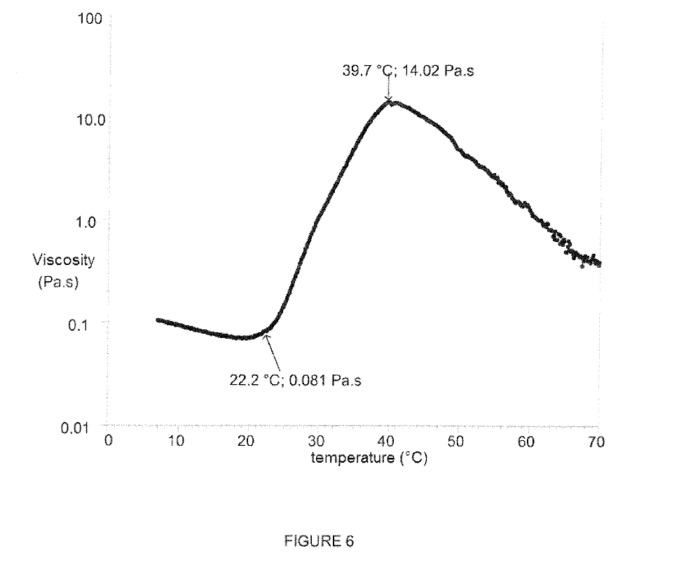

The flow viscosity curve under shear at 10 s⁻¹ of the composition of Example 6 as a function of temperature is shown in FIG. 6.

### EXAMPLE 7

The following anti-snoring composition was prepared:

| EG 230 (ExpertGel ®) | 3.5 | g | 7% | |
|---|---|---|---|---|
| Ethanol | 2.5 | g | 5% | |
| Hyaluronic acid | 0.1 | g | 0.2% | |
| Chlorhexidine | 0.05 | g | 0.1% | |
| Sodium chloride | 0.45 | g | 0.9% | |
| Menthol | 0.05 | g | 0.1% | |
| Tween 20 ® | 0.15 | g | 0.3% | |
| Mineral water | 43.2 | g | q.s. | 100% |

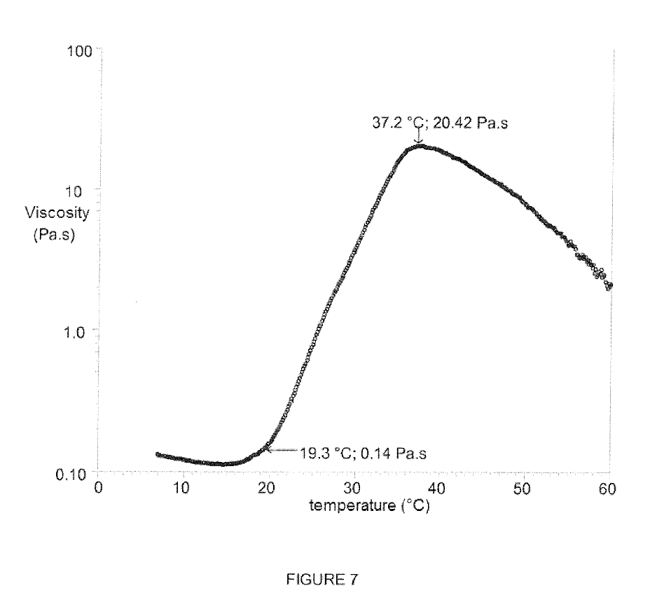

The flow viscosity curve under shear at 10 s⁻¹ of the composition of Example 7 as a function of temperature is shown in FIG. 7.

### EXAMPLE 8

### Study of the Release of an Active Ingredient from a Formulation Containing a Thermogelling Polymer

An active ingredient that has received particular study is DL-lysine acetylsalicylate (Aspégic®). The formulation presented in Table 1 below was prepared with an aqueous solution of ExpertGel® polymer No. EG56,

The thermogelling polymer EG56 (CAS number 93665-35-1) is a branched polyurethane containing PEO-b-PPO-b-PEO units, the INCI (International Nomenclature of Cosmetic Ingredients) designation of which is BIS-Methoxy PEG-13 PEG-438/PPG-110 SMDI Copolymer.

TABLE 1

| | | | [Aspégic ®] |
|---|---|---|---|
| Thermo- | | | maximum in |
| gelling | [ExpertGel ®] | [Aspégic ®] | water at |
| polymer | % w/w | % w/w | 37° C. |
| ExpertGel ® | formulation | formulation | g/L |
| EG56 | 7.50 | 10 | 2.34 |

This solution was then added dropwise to 20 ml of water at 37° C. The drops gel immediately on contact with water at 37° C. and coat the active ingredient.

The kinetics of release of Aspégic® at 37° C. was monitored by UV-visible spectroscopy.

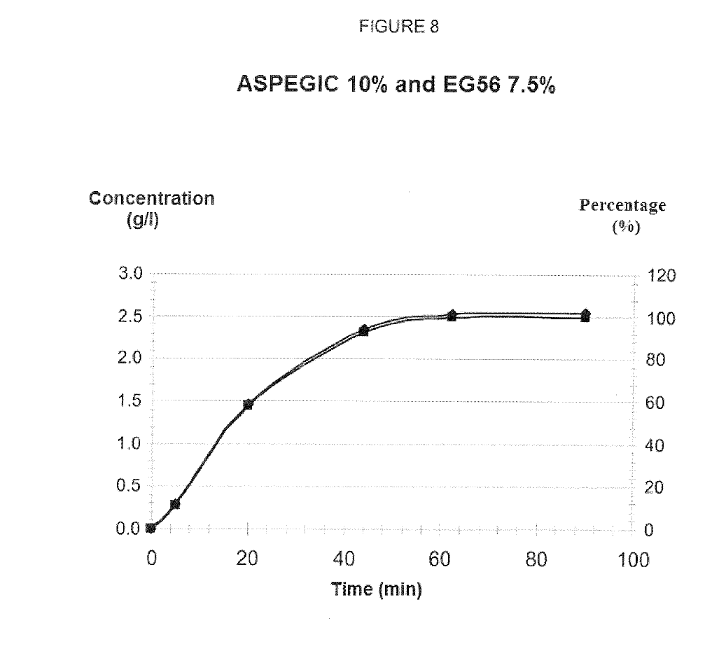

The curve in FIG. 8 shows the release kinetics at 37° C. of Aspégic® encapsulated in a solution of EG56C at 7.5% in water at 37° C., expressed on the ordinate on the left (symbol -♦-) as concentration (g/l), and on the ordinate on the right (symbol -▪-) as percentage by weight (%), as a function of time (min).

Table 2 presents the release kinetics of Aspégic®, free or in an aqueous solution of thermogelling polymer.

TABLE 2

| | Time for releasing | Time for releasing |
|---|---|---|
| Solution tested | 50% of Aspégic ® | 100% of Aspégic ® |
| 7.5% EG56 + 10% | 16 min | 62 min |
| Aspégic ® | | |
| Solution of 10% | 0 | 0 |
| Aspégic ® | | |

The results show that the use of thermogelling polymer makes it possible to control the release of the active ingredient.

1. (canceled)

2. The method of claim 16, wherein said thermogelling polymer displays, in aqueous solution, a viscosity below 0.1 Pa·s under 10 s⁻¹ of shearing at room temperature and a viscosity above 0.1 Pa·s, under 10 s⁻¹ of shearing in contact with the mucosae, at body temperature.

3. The method of claim 16, wherein said thermogelling polymer is present at a concentration by weight from 2 to 10% in the aqueous solution.

4. The method of claim 16, wherein said thermogelling polymer is selected from block polymers and graft polymers.

5. The method of claim 16, wherein said units having a lower critical solution temperature in water are selected from polyethers, N-substituted derivatives of acrylamide, polyvinylcaprolactam and copolymers of vinylcaprolactam.

6. The method of claim 16, wherein said thermogelling polymer is selected from polyurethanes having poly(ethylene oxide-b-propylene oxide-b-ethylene oxide) (PEO-b-PPO-b-PEO) groups.

7. The method of claim 16, wherein said thermogelling polymer is selected from polyurethane having poly (ethylene oxide-b-propylene oxide-b-ethylene oxide) (PEO-b-PPO-b-PEO) groups and is obtained by polycondensation of diisocyanates and of triblock dials PEO-b-PPO-b-PEO and can comprise urea groups and/or allophanates.

8. The method of claim 16, wherein said composition further comprises an active ingredient for controlled release.

9. The method of claim 16, wherein said composition further comprises an active ingredient selected from a decongestant, an anti-inflammatory, an agent for treating or preventing snoring and a vasoconstrictor.

10. The method of claim 16, wherein the composition further comprises at least one additive selected from electrolytes, lubricants, alcohols, non-ionic surfactants, and polysaccharides.

11. The method of claim 16, wherein administering is conducted by spraying and said composition is suitable for spraying, by the nasal or buccal route.

12. The method of claim 16, wherein said composition has muco-adhesive properties, enabling it to resist being washed away by a flow of saliva.

13. The method of claim 16, wherein said composition displays properties of lubrication of the mucosae.

14. (canceled)

15. (canceled)

16. A method for preventing and/or treating snoring comprising:
administering to an individual in need of preventing and/or treating snoring a composition comprising at least one thermogelling polymer in an aqueous solution, said thermogelling polymer comprising water-soluble units and units having a lower critical solution temperature in water and said thermoyelling polymer present at a concentration of less than or equal to 10% by weight in said aqueous solution.
